(19) United States
(12) Patent Application Publication
(10) Pub. No.: US 2018/0286514 A1
(43) Pub. Date: Oct. 4, 2018

(54) COMPUTERIZED SYSTEM, METHOD AND GRAPHICAL USER INTERFACE (GUI) FOR PREDICTION, DISPLAY AND COMPARISON OF PROBABILITIES OF SUCCESS AND COMPLICATIONS OF EXTRACORPOREAL SHOCKWAVE LITHOTRIPSY (ESWL) AND URETEROSCOPY (URS) FOR SURGICAL MANAGEMENT OF STONE DISEASE

(71) Applicant: Translational Analytics and Statistics, LLC, Tucson, AZ (US)

(72) Inventors: Ryan Geoffrey Nathaniel Seltzer, Tucson, AZ (US); Christopher Mark Gleason, Tucson, AZ (US); Blake Douglas Hamilton, Holladay, UT (US)

(21) Appl. No.: 15/899,984

(22) Filed: Feb. 20, 2018

Related U.S. Application Data
(60) Provisional application No. 62/478,314, filed on Mar. 29, 2017.

(51) Int. Cl. G16H 50/20; G16H 10/60

(52) U.S. Cl. CPC G16H 50/20; G16H 10/60

(57) ABSTRACT

A GUI driven computer implemented system prediction, comparison and selection of treatment modalities for surgical management of stone disease. A computer memory is configured to store regression weights for stone, patient, machine prediction variables for ESWL and URS for a percentile grouping calculation. A computer processer configured to compute a probability of success (Ps) and a probability of complications (Pc) for the ESWL and URS treatment modalities for the percentile grouping as a weighted combination of the regression weights and user input values for the prediction variables. The GUI includes a user selectable display space configured to display the stone, patient and machine prediction variables and receive and display user input of values for each prediction variable and a first results display space configured to display the computed Ps and Pc for each of ESWL and URS for the percentile grouping.

### CROSS-REFERENCE TO RELATED APPLICATIONS

This application claims benefit of priority under 35 U.S.C. 119(e) to U.S. Provisional Application No. 62/478,314 entitled “Computerized System, Method and Graphical User Interface (GUI) for Prediction, Display and Comparison of Probabilities of Success and Complications of Extracorporeal Shockwave Lithotripsy (ESWL) and Ureteroscopy (URS) for Surgical Management of Stone Disease” and filed on Mar. 29, 2017, the entire contents of which are incorporated by reference.

### BACKGROUND OF THE INVENTION

### Field of the Invention

This invention relates to the surgical management of stone disease (“nephrolithiasis”) and more particularly to a system, method and graphic user interface (GUI) for the prediction, display and comparison of probabilities of success and complications of extracorporeal shock wave lithotripsy (ESWL) and ureteroscopy (USR) to each other and literature benchmarks to enable informed selection of the most effective treatment modality.

### Description of the Related Art

### Urolithiasis

The exact reasons of stone formation are not known; however, diet and hydration are thought to play a critical role. Symptomatic urinary track calculi will affect one in ten Americans. The increasing rates of obesity, type II diabetes and metabolic syndrome have all been associated with increased stone formation. Additionally, the more severe the type II diabetes, the greater the risk of urolithiasis.

Urolithiasis refers to the presence of calculi (stones) in the urinary system. Stones form in the urinary tract by the deposit of crystalline substances (calcium oxalate, calcium phosphate, uric acid, cystine) excreted in the urine. They may be found anywhere from the kidney to the bladder and vary in size from minute granular deposits called sand or gravel, to bladder stones the size of an orange. Heredity, age, sex, geographic location, environmental temperature, water intake, diet, social class and occupation of the individual all play some part in the genesis of urinary calculi.

### Ureteroscopy (URS)

As illustrated in FIG. 1, direct visualization into the lumen of various portions of the urinary tract 100 with a telescopic instrument 102 is a routine part of the practice of Urology. A variety of either flexible or rigid endoscopes (cystoscopies, ureteroscopes, and nephroscopes) are available to inspect various portions of the urinary tract. Once a stone 104 is visualized endoscopically, a variety of techniques can be used to treat it. Endoscopic lithotripsy is the shattering of a calculus in the urinary tract by passing a probe through the endoscope. These methods require placing a fiber or probe through an endoscope to place very close to or touching the stone with visualization of the stone:
A. Ultrasonic lithotripsy uses ultrasonic waves transmitted through a probe to vibrate the stone. Several thousand mechanical vibrations a second are used to fragment the stone as the probe is placed against it.
B. Electrohydraulic lithotripsy (EHL) places a probe near the stone generating multiple hydraulic shock waves created by electrical discharges that shatter the stone.
C. Laser lithotripsy delivers laser energy to the surface of the stone via a quartz laser fiber. The pulsed dye laser emits energy causing stress fractures that lead to stone fragmentation. As a precaution the patient as well as the staff personnel must wear wavelength specific protective glasses or goggles to protect the eyes.

The most common procedure utilizes a Holmium Laser, which uses a flash lamp to excite a Holmium crystal, emitting laser energy at a wavelength of 2100 nanometers transmitted through a flexible fiber. As a result of the superheating of fluid at the fiber tip and the creation of microscopic vaporization bubble, stone fragmentation occurs. The fiber tip must be in contact with the stone for fragmentation due to the shallow penetration of the laser energy.

### Extracorporeal Shock Wave Lithotripsy (ESWL)

As illustrated in FIG. 2, ESWL is a noninvasive treatment for urinary tract calculi using a lithotripter device 200. The lithotripsy device generates and focuses a shock wave 202 on the stone's surface causing the stone to fragment. These fragments then pass with urine out of the body. ESWL may be used in all locations of the urinary tract. A patient 204 is supported by a water or gel-filled cushion 206 on a table 208. A few of the different type of ESWL machines and their features are:
A. Storz SLX-T: Electromagnetic shockwave generation with a focal point of 28×6×6 mm. Can use any mobile x-ray c-arm. Requires a motorized pallet jack to move the table. Reliable. Moderately expensive compared to others partially depending on the c-arm chosen
B. Storz F2: Electromagnetic shockwave generation with two focal point choices. One is 20×2×2 mm and the other is 36×5×5. Can be used with any c-arm or can be purchased with an integrated version. Comes with motorized controls for movement. Reliable but more expensive then the SLX-T.
C. Dornier Compact Delta: Spark gap shockwave generation with a focal point of 80×7×7. Comes with an integrated c-arm and is the most mobile of the 3. Least expensive with good reliability.

Providing ESWL services requires significant preparation. While there are a variety of lithotripters on the market, they all require roughly the same set-up. These steps include:
Unloading, delivering and decontaminating the equipment for use in the operating room. This often includes a mobile x-ray unit 210 including an X-ray source 212 and an X-ray receiver 214, the lithotripter 200 and ancillary equipment such as monitors 216 needed for the case.
Performing facility required electrical and safety inspections.
Calibrating the x-ray unit to the shockwave therapy head per manufactures guidelines.
Testing all the equipment for operational readiness.

An ESWL procedure usually takes 30-60 minutes to complete depending on the details of the treatment. Every procedure starts with an evaluation of the patient's health, diagnoses and a check for any possible contraindications for treatment. The patient is then placed under anesthesia and positioned for the ESWL. The lithotripsy technologist localizes the stone using x-ray and/or ultrasound and targets the stone within the shockwave of the lithotripter. Treatment energy levels and rates are manipulated by a technologist 218 under the direction of the treating urologist. The patient and the stone are closely monitored throughout the procedure and the treatment parameters are adjusted accordingly. Completion of the procedure is achieved a couple different ways. First, if the stone fractures so well and is no longer visible (and there are no other stones to treat) then the procedure is done. Second, if the maximum shock count is reached. Maximum shock counts are put in place by the manufactures to protect patients. ESWL is minimally invasive but still causes trauma to the patient and must be mitigated.

In appropriately selected patients, the overall success rate of extracorporeal shockwave lithotripsy (ESWL) is higher than 90% for stone clearance, with patients remaining stone-free for up to 2 years. Compared with ureteroscopic removal of stones, ESWL leads to less complications and shorter hospital stays. In less than 0.1% of cases, clinically significant bleeding around the kidney may occur resulting in a perirenal hematoma that requires treatment. However, advances in ureteroscopy and difficulty in properly selecting patients have seen the gap narrow. Some recent studies have shown ureteroscopy to achieve greater stone-free rates. This continues to be a point of debate with the Urological community.

### SUMMARY OF THE INVENTION

The following is a summary of the invention in order to provide a basic understanding of some aspects of the invention. This summary is not intended to identify key or critical elements of the invention or to delineate the scope of the invention. Its sole purpose is to present some concepts of the invention in a simplified form as a prelude to the more detailed description and the defining claims that are presented later.

The present invention provides a graphical user interface (GUI) driven computer implemented system for prediction, comparison and selection of treatment modalities for surgical management of stone disease configured for display on a display screen of an electronic device.

In an embodiment, the GUI driven computer implemented system comprises computer memory configured to store regression weights for a plurality of stone prediction variables for each of a extracorporeal shock wave lithotripsy (ESWL) and a Ureteroscopy (URS) treatment modality and a computer processer configured to compute a probability of success (Ps) and a probability of complications (Pc) for the ESWL and URS treatment modalities as a weighted combination of the regression weights and values for the prediction variables. Instructions stored in the computer memory are executable by the computer processor to display and operate a GUI. The GUI comprises at least one user selectable display space and at least one results display space configured for simultaneous display on the display screen of the electronic device. A first user selectable display space is configured to display the plurality of stone prediction variables and receive and display values for each stone and patient prediction variable. A first results display space is configured to display the computed Ps and Pc for each of ESWL and URS calculations.

In different embodiments, the stone prediction variables include at least stone size and may further include stone density and stone location. Patient prediction variables may include BMI, anesthesia, sex, age, medical history, pre-op procedures etc. Other prediction variables may include a make and model of an ESWL machine or URS device.

In different embodiments, the regression weights may be scalar values, a function of other prediction variable values or conditional probabilities from a Bayesian model. The weights may be configured for linear or non-linear combinations of the prediction variables.

In different embodiments, Ps and Pc are computed and displayed for the ESWL and URS treatment modalities for both a benchmark and a percentile grouping. These sampling cohorts may also be broken down by stone size categories.

In different embodiments, a second user selectable display space is configured to display a calculate button, user selection of which initiates the computer processor to compute Ps and Pc.

In different embodiments, the first user selectable display space is configured to receive user input of the values for the prediction variables.

In different embodiments, the regression weights are determined from data analytics and verified against published literature and clinical assessment. An intercept in the regression equation accounts for differences in successful outcomes between different benchmark and percentile groups, and may include additional Ps and Pc calculated from scientific literature only for additional comparison. If the relationship between predictor variables and outcomes is expected to differ by benchmark or percentile groups (i.e., differences not just due to intercept), then variable regression weights may also account for differences among benchmark and/or percentile groups.

In different embodiments, the single, final regression weight for each input variable obtained from the literature will be estimated using a formal meta analysis.

In different embodiments, the single, final regression weights obtained from data analysis will be blended with the regression weights from the literature by adding the data analysis regression weights to the calculations of the literature meta analysis, so that the data analysis regression weights are used as another study on which the meta analysis is based (i.e., the meta analysis is conducted on all of the regression weights reported in the literature and the regression weights obtained in data analysis). This process ensures that the regression weights from data analysis are empirically blended with the weights reported in the peer-reviewed literature.

In an embodiment, the computer processor is configured to calculate and display via the GUI confidence intervals for each of the Ps and Pc values. In different embodiments, the Ps and Pc are displayed as bars on a bar graph and the confidence intervals are displayed as error bars on top of each bar.

In an embodiment, the computer processor is configured to calculate and display via the GUI confidence intervals and actual Ps and Pc values for different percentile groups, such as the Ps and Pc for the 90th or 75th percentile.

In an embodiment, the computer processor is configured to calculate and display economic and general cost considerations between ESWL and URS treatment modalities. Such economic and general cost variables may be equipment cost, operating room time, procedure reimbursement, repeat procedures, and length of hospital stay.

In an embodiment, the computer processor is configured to calculate and display probability of complications broken out by complication type (e.g., bleeding, infection, and pain).

These and other features and advantages of the invention will be apparent to those skilled in the art from the following detailed description of preferred embodiments, taken together with the accompanying drawings, in which:

### BRIEF DESCRIPTION OF THE DRAWINGS

FIG. 1, as described above, illustrates a ureteroscopy (URS) procedure;

FIG. 2, as described above, illustrates an extracorporeal shock wave lithotripsy (EWSL) procedure;

FIG. 3 is a diagram of a GUI driven computer implemented system for prediction, comparison and selection of ESWL and URS treatment modalities for surgical management of stone disease;

FIGS. 4a-4g are a sequence of screen shots of the GUI for prediction, comparison and selection of ESWL and URS treatment modalities;

FIG. 5 is a table of an embodiment of stone, patient and machine prediction variables;

FIGS. 6a and 6b are an embodiment of a regression weights table; and

FIG. 7 is an embodiment of a method for defining the regression equation and determining the regression weights and intercept.

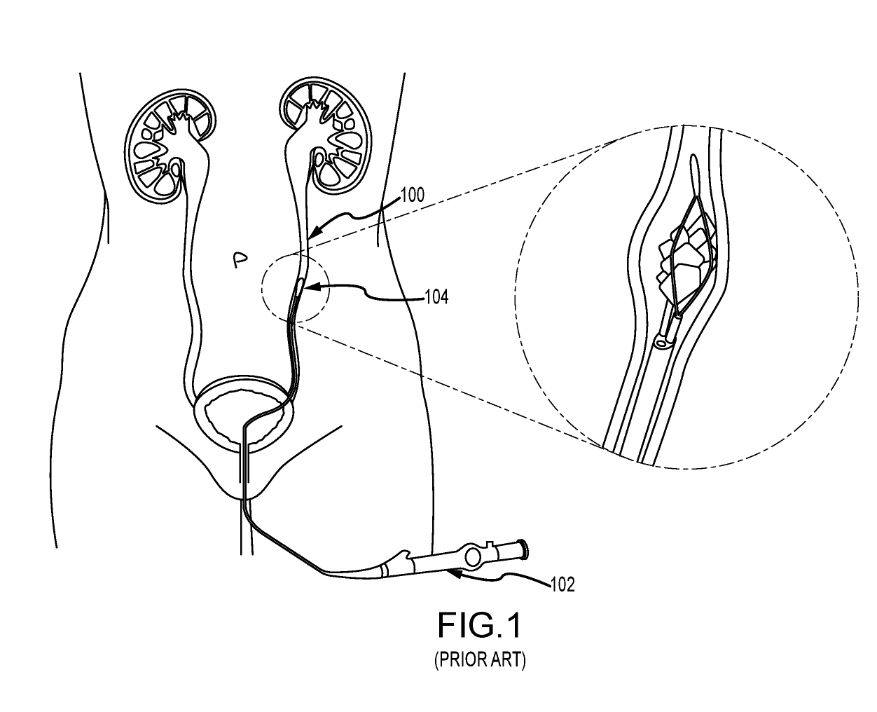

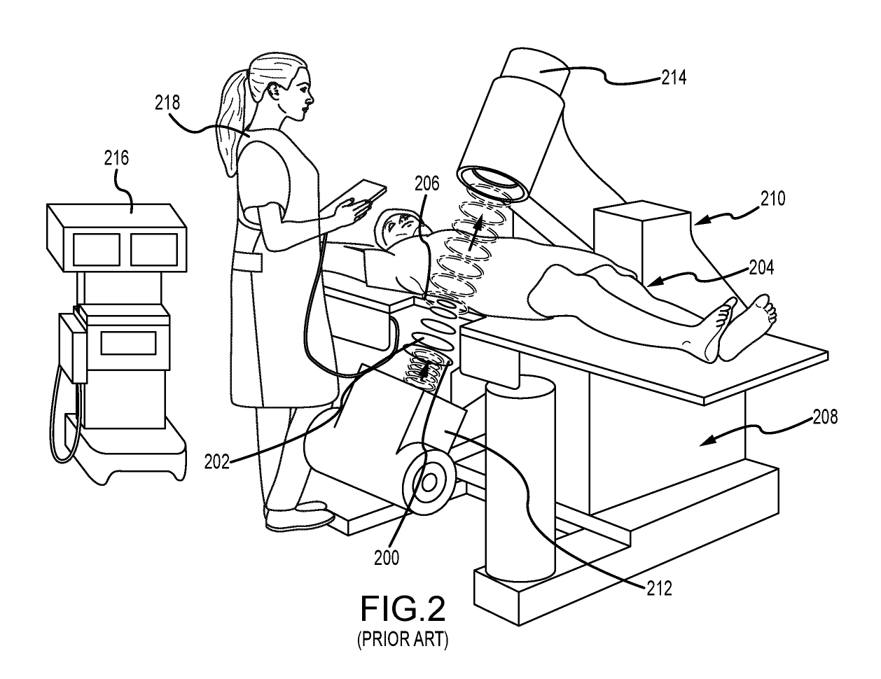

### DETAILED DESCRIPTION OF THE INVENTION

Success in treating stone disease is based on improving or optimizing the likelihood of fragmenting a stone completely or small enough that the stone will pass on its own, minimizing complications due to the treatment procedure and making procedures more cost effective given that ESWL can have higher reimbursement rates, lower operating room time, and lower disposable equipment costs compared to URS. This optimization of stone fragmentation and treatment complication is mostly dependent on selecting both the treatment modality (ESWL, URS, etc.) and treatment strategy (shock rate, x-ray frequency, maximum power level, etc.) that align with the physical characteristics of the patient (BMI, medication use, etc.) and stone (size, density, location, etc.). Therefore, to optimize desired outcomes, the urologist must know which treatment modality and treatment strategy is best suited for a particular patient and stone profile.

Currently, the selection of treatment modality and strategy is most frequently based on treating urologists' experiences, training, personal preferences, etc. Decisions based on these potentially biased sources may threaten a urologist's ability to accurately make treatment choices that optimize stone fragmentation and treatment complication.

One of the reasons that urologists may not be able to optimize treatment when basing decisions on experience and previous training is that they may not be either aware of all of the relevant factors that contribute to optimization of treatments or realize the relative importance of specific treatment elements in determining the desired outcome or optimization (e.g., the effect of a particular decision on the treatment outcome). This lack of knowledge prevents them from being indifferent to the optimal modality or a particular treatment strategy when making fine-grained treatment decisions, and they end up making decisions based on experience and personal preference. To effectively create a treatment plan to optimize stone fragmentation and complication, urologists need to know all of the relevant factors that influence a treatment and know which levels or values of each factor are ideal for a given patient and stone profile.

The information needed to optimize treatment plans for specific, fine-grained patient and stone profiles (e.g., 8 mm stone in upper ureter for 55 year old, male patient with a BMI of 23) needs to be in part based on data analytics. This is because data analytics are able to compile and analyze a large number of variables and conditions that need to be simultaneously considered when creating a treatment plan. The resulting treatment plans that consider fine-grained patient and stone characteristics can lead to higher success rates, profits, and patient satisfaction, as well as lower complication rates compared to treatment plans based on personal preference.

To address the fact that doctors need to identify relevant variables that affect outcomes and know the values and decisions for each of these variables, regression equations are provided that produce a set of predicted probabilities including stone fragmentation (Ps) and treatment complication (Pc) and possibly predicted infection rate (Pi), predicted hematoma rate (Ph), predicted pain rate (Pp), predicted operating room time (Po), and Predicted stent rate (Pn) based on patient and stone characteristics. Furthermore, a graphical user interface (GUI) is provided that will engage urologists, technologists, and patients in producing these predicted probabilities for real or hypothetical patients. The user can enter patient and stone characteristics and can see the predicted probabilities (and possibly confidence intervals) of stone fragmentation Ps, complication Pc, and others in the set of predicted probabilities for ESWL and URS. These probabilities may be displayed for benchmark and/or percentile groupings. Benchmark groups may be calculated from academic literature or from data registries, and percentile groupings, such as 90th percentile performers in terms of success rate, may be calculated from data registries of, if available, the academic literature. Together the regression engine and GUI form a Stone Decision Engine to drive prediction, comparison and selection of the optimal (increase treatment success and minimize complications) treatment modality for surgical management of stone disease.

The regression equation and GUI can improve a urologist's knowledge directly by identifying the relevant elements involved in performing a treatment and making decisions, and then providing recommendations (based on probability of outcomes) for how and when to incorporate these elements into practice. This interactive feature is intended to engage and educate users to the relevant elements or variables that affect outcomes in stone disease treatment. In addition, this engagement will help urologists internalize and integrate this information into their practice by increasing the meaning, motivation, and engagement of using tools to learn and adopt best practices. For example, while many urologists may have heard the benefit of a certain treatment activity or machine, they may not internalize the impact of this activity until they see how it relates to desired outcomes.

With the information presented in this program, users may create an overall strategy for treating stone disease and standardize and align their practices and protocols with American Urological Association (AUA) recommendations, peer-review publications, and results of big data analytics. The creation of evidence based strategies will happen by having practitioners better weigh patient and stone demographics for making a treatment decision, better understand the relevant variables that affect treatment outcomes (when decisions need to be made), better evaluate how to perform the treatment (which decisions lead to better outcomes), and better understand the outcomes of each decision. These actions can lead to more effective treatments with better stone fragmentation, safer treatments with lower complication rates, and more profitable treatments with optimized treatment strategies and equipment use.

This program can keep practitioners continually updating their actions to the most current evidence-based recommendations and using all relevant information when making decisions. Educators, facilities, insurance companies, and practicing urologists will all find benefit in the application, dissemination, and enforcement of evidence-based protocols into patient care.

Ultimately, the Stone Decision Engine will help users augment their professional experience to select the most appropriate treatment modality for a given patient and stone profile so that decisions that optimize effectiveness and safety are based on scientific data. These improved decisions and adherence to protocols and best practices can increase success rate, decrease morbidity and cost, and strengthen the identity of lithotripsy companies in the market (by their use of data-driven translational processes, analytics, and improved outcomes).

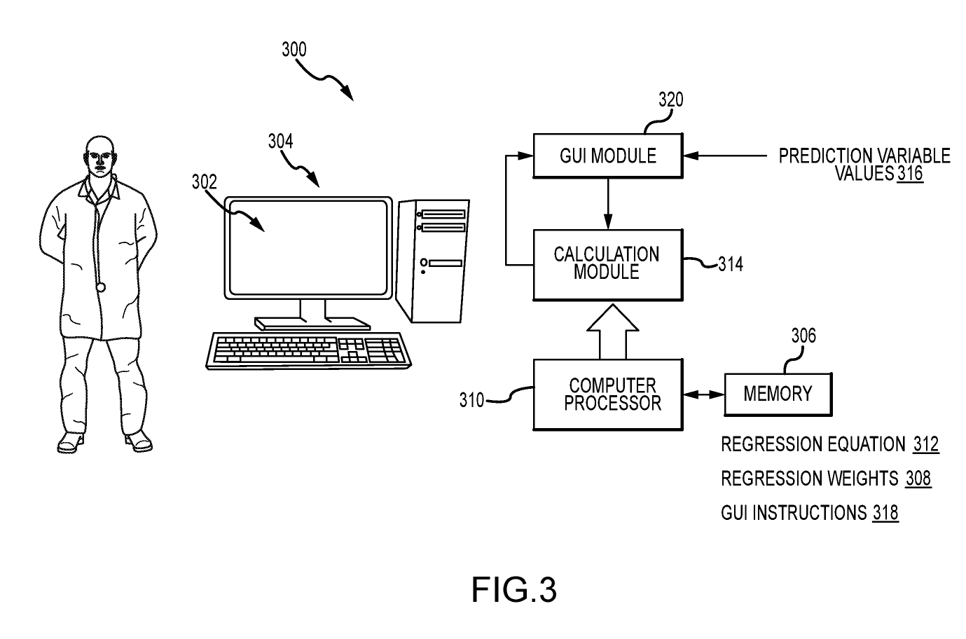

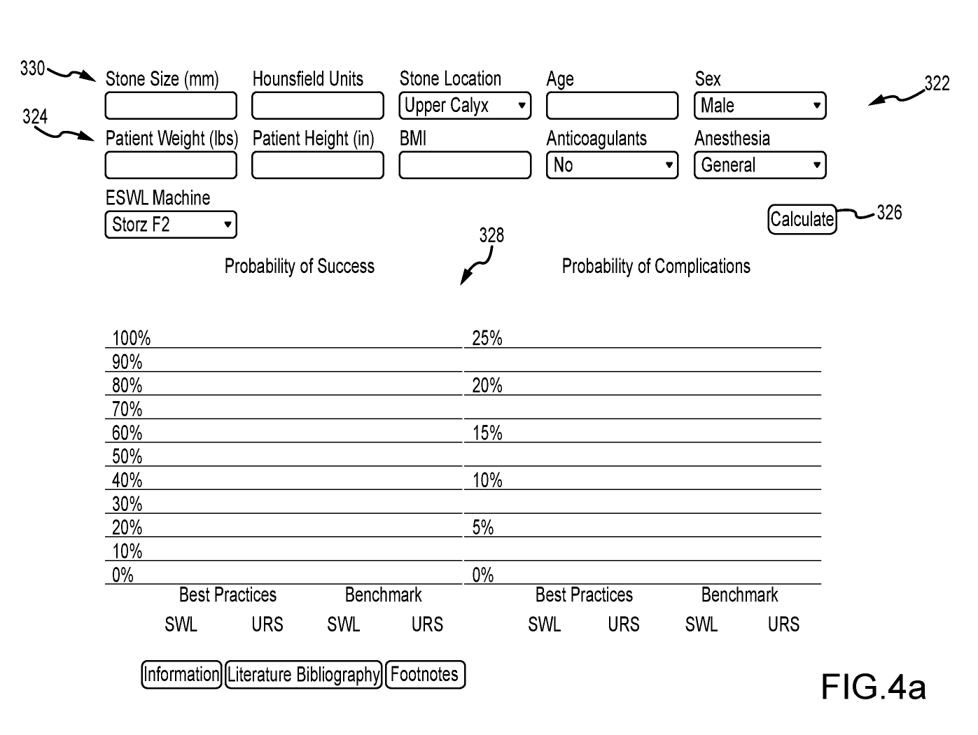

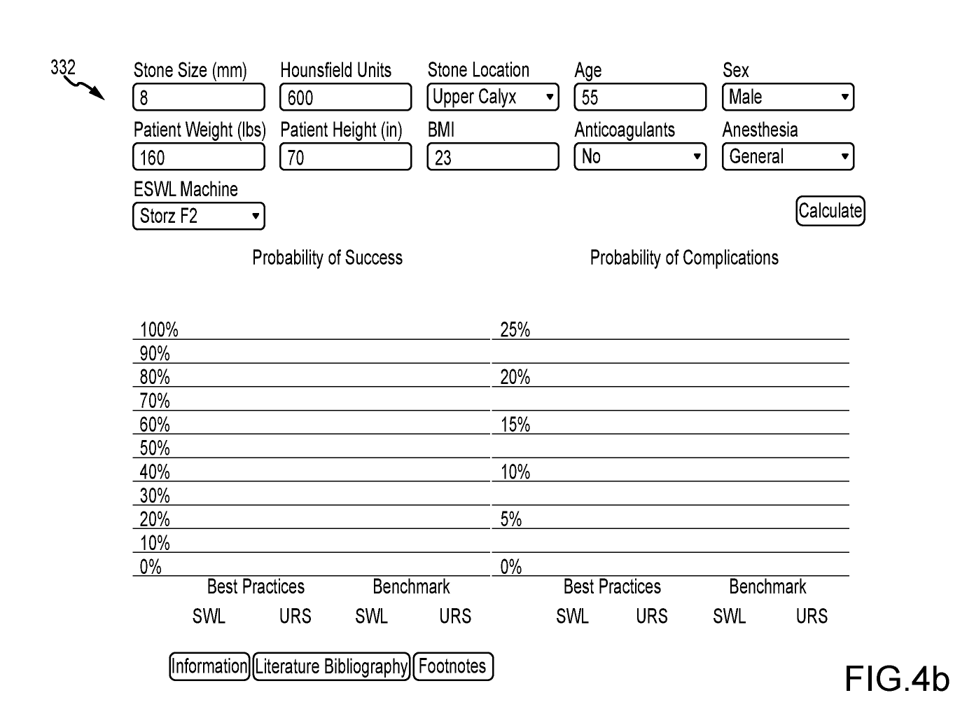

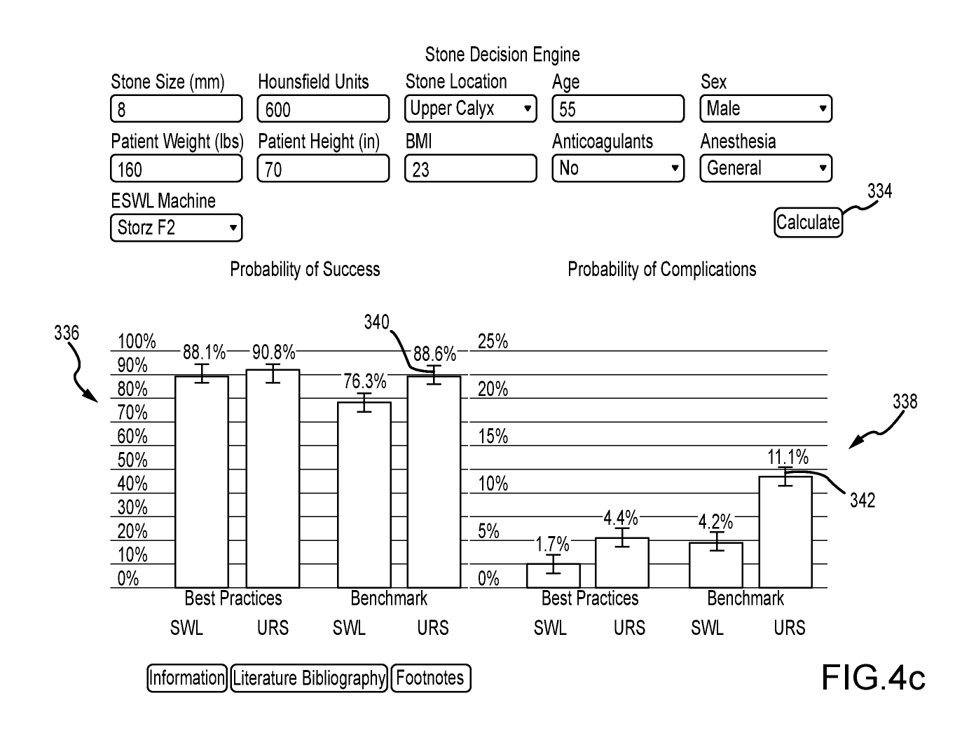

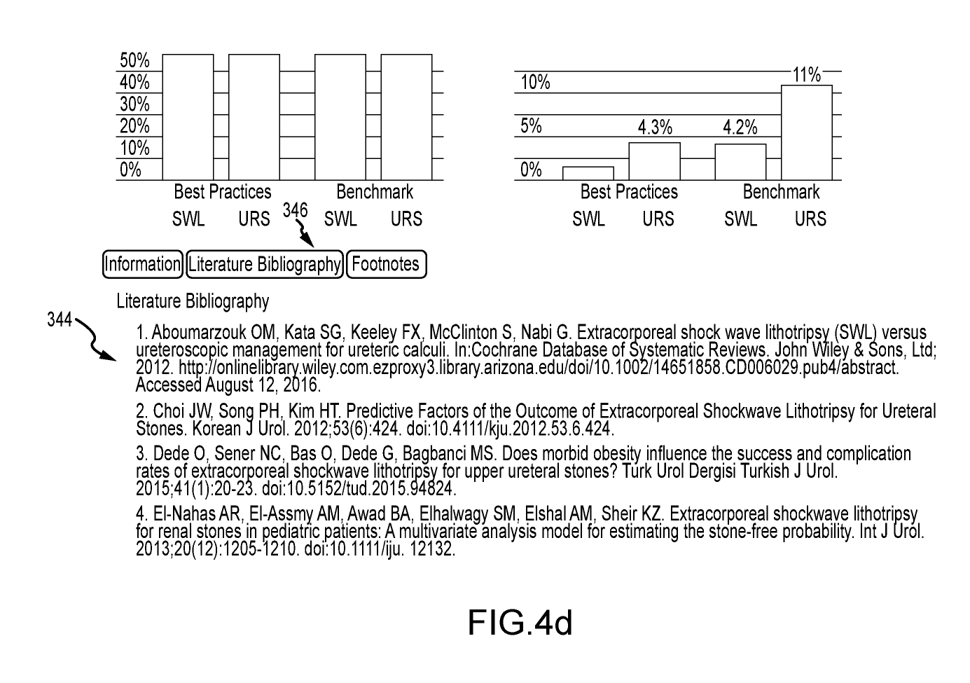

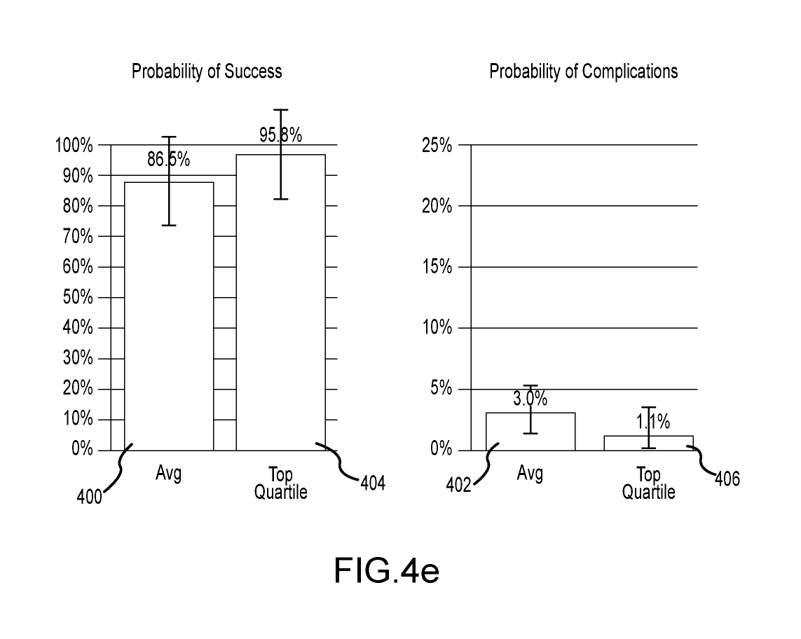

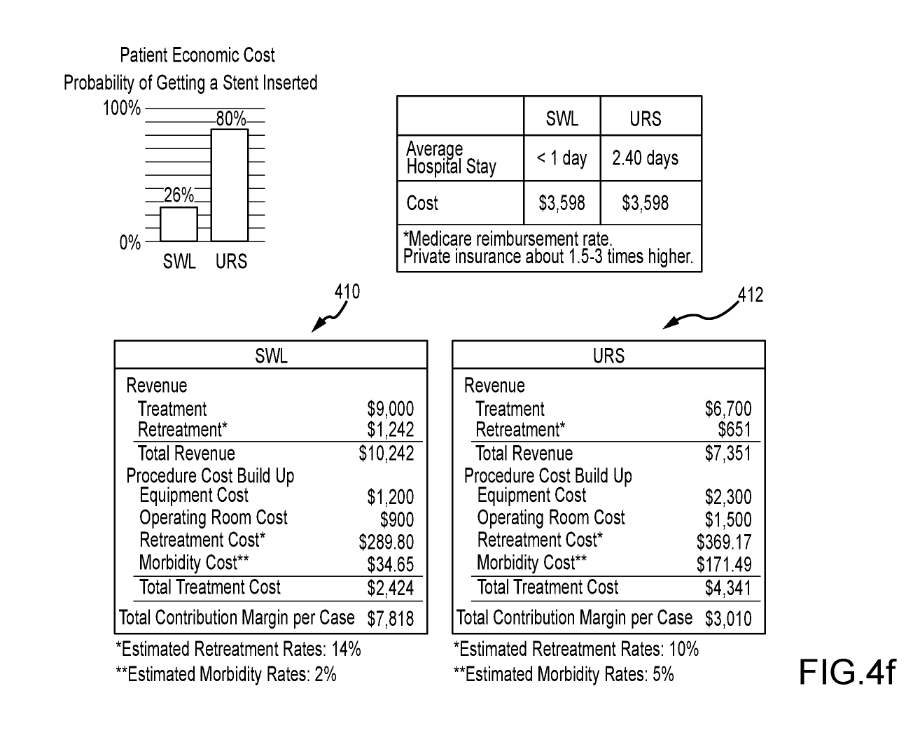

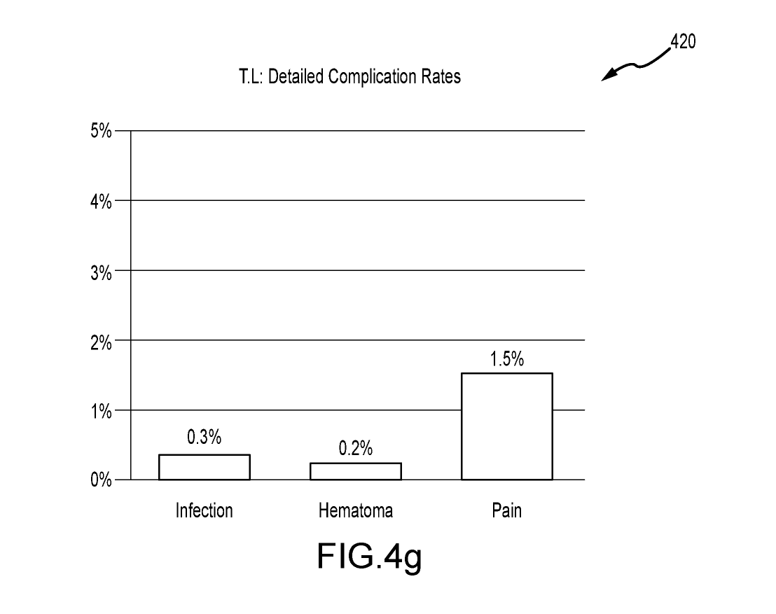

Referring now to FIGS. 3 and 4a-4g, an embodiment of a graphical user interface (GUI) driven computer implemented system 300 is configured to implement a regression equation for prediction, comparison and selection of treatment modalities for surgical management of stone disease and configured for display on a display screen 302 of an electronic device 304 such as a desktop computer, laptop, tablet or other hand-held device. Together the GUI and regression equation define the Stone Decision Engine to guide the users' selection of the ESWL or URS treatment modality best suited for a particular patient treatment based on specific characteristics of that patient and that patient's stone.

In an embodiment, the GUI driven computer implemented system 300 comprises computer memory 306 configured to store regression weights 308 for at least a plurality of stone prediction variables and possibly patient and machine prediction variables for each of an extracorporeal shock wave lithotripsy (ESWL) and a Ureteroscopy (URS) treatment modality for both a benchmark and percentile groupings calculation and a computer processer 310 configured to execute a regression equation 312 to compute via a calculation module 314 a probability of success (Ps), probability of complications (Pc), and the other calculations in the set of predicted probabilities (e.g., Pi, Pn, etc.) for the ESWL and URS treatment modalities for benchmark and percentile groupings as a weighted combination of the regression weights and user input values 316 for the prediction variables. Instructions 318 are stored in the computer memory executable by the computer processor via GUI module 320 to display and operate a GUI 322 on display screen 302.

The GUI 322 comprises a first user selectable display space 324 and at least one results display space 328 configured for simultaneous display on the display screen 302 of the electronic device 304. The first user selectable display space 324 is configured to display the plurality of stone, patient and machine prediction variables 330 and receive and display values (or selections) 332 for each prediction variable. Values for prediction variables may be inputted manually by typing in values or selecting pull down options, by porting in some or all values from an external source (e.g., another program or database where the same info has been generated), or from pre-programmed default values. A second user selectable display space 326 is configured to display a calculate button 334 (or equivalent), user selection of which initiates the computer processor 310 to execute the regression equation to compute the Ps and Pc. Alternately, the GUI may be configured to automatically initiate computation when values are entered in each of the fields. The results display space 328 is configured to display the computed Ps 336 and Pc 338 for each of ESWL and URS for both the benchmark and best practices calculations. As shown, Ps and Pc are depicted as vertical bars in a bar graph. Alternate techniques may be used to display the Ps and Pc values.

The computer processor 310 may be configured to compute and overlay confidence intervals 340 and 342 for Ps, Pc, and other values in the set of predicted probabilities (e.g., Pi, Pn, etc.). In an embodiment, confidence intervals are calculated with the following equation: β=±1.96(SE)(β), where β is the regression weight, 1.96 is a fixed value of the 97.5^{th} percentile of the standard normal distribution (Rabe-Hasketh and Skrondal (2008), and SE is the standard error. Alternate techniques may be used.

The literature bibliography 344 used to form the benchmark may be displayed via a button 346. Providing a record of and access to the bibliography is useful because it allows users to see and evaluate the peer-reviewed publications that were used in creating the benchmark numbers. Providing this information lends credibility to the methods used in calculations using scientific literature.

In an embodiment, the computer processor can be configured to calculate and display via the GUI confidence intervals and actual set of predicted probability values (e.g., Ps, Pc, Pi, Pn, etc.) for different percentile performers, such as the Ps and Pc for the 50^{th} (average) and 75^{th} (top quartile) percentile 400, 402 and 404, 406 as shown in FIG. 4e. These percentiles can be calculated by finding the intercept that produces the observed success rate for a certain percentile in the data registry used for analysis or by recreating the algorithm using only members, such as urologists, in a database that comprise the select percentile. This latter method may be used to support hypotheses that predictor variable-outcome relationships actually change by percentile category (i.e., the relationship of stone size on treatment success is actually different for 90^{th} percentile urologists than for 50^{th} percentile urologists). These percentile groupings may also be broken out by stone size categories.

In an embodiment, the computer processor is configured to calculate and display economic and general cost considerations between ESWL and URS treatment modalities 410 and 412 as shown in FIG. 4f. Such economic and general cost variables may be equipment cost, operating room time, procedure reimbursement, repeat procedures, and length of hospital stay, and are calculated using the set of predicted probabilities (e.g., Pi, Pn, etc.). The economics of a treatment modality may affect the Doctor's or patient's selection of an appropriate treatment.

In an embodiment, the computer processor is configured to calculate and display probability of complications 420 broken out by complication type (e.g., bleeding, infection, and pain) as show in FIG. 4g.

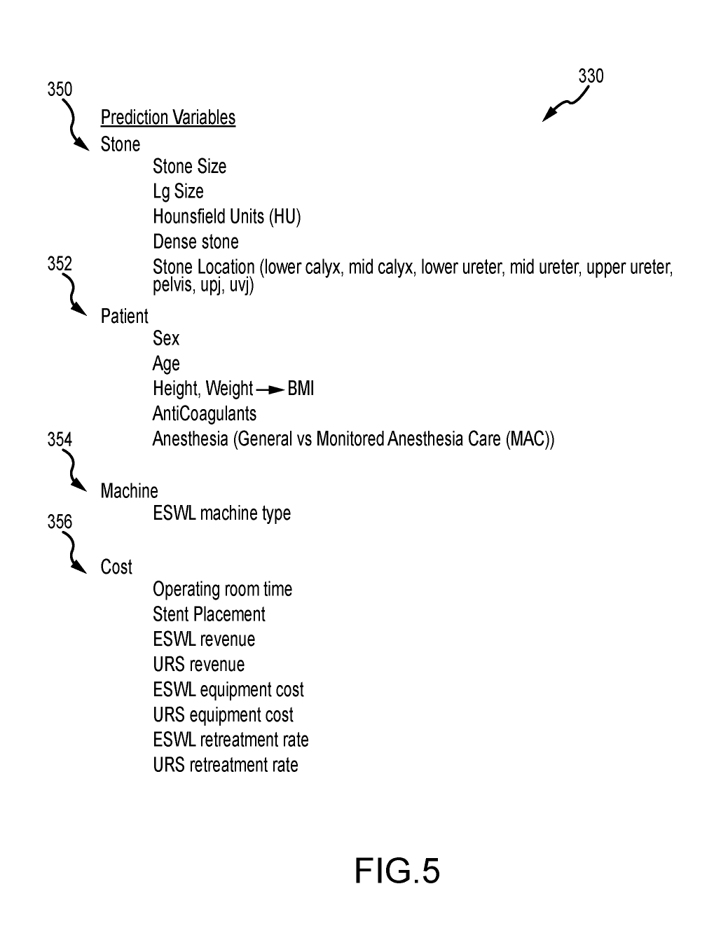

Referring now to FIG. 5, the prediction variables 330 include a number of stone, patient, machine, and cost prediction variables 350, 352, 354, and 356, respectively. These may include variables whose inputs are directly input or selected by the user, pre-populated or default values, or variables whose inputs are derivative of inputs for other variables. For example, Lg Size is derivative of Stone Size and Dense Stone is derivative of HU.

The set of prediction variables is defined to include variables that both contribute to overall calculation (and accuracy) of the Ps, Pc, and other P in the set of predicted probabilities (e.g., Pi, Pn, etc.) values and that discriminate between the ESWL and URS treatment modalities. For example, stone size and stone density are key factors in both the overall Ps and Pc for both treatment modalities as well as discriminating between treatment modalities. URS tends to provide a better Ps for large and/or dense stones. Similarly, URS tends to outperform ESWL for larger BMI. However, URS in general tends to have a higher Pc than ESWL and specifically may be more sensitive to stone location.

At a minimum, the regression equation includes stone prediction variables 350, and in particular at least a stone size (“stone size”) prediction variable. The Stone Size variable is typically the diameter of the stone in mm. A stone density variable (“HU”) may also be included. HU or Hounsfield Unit is a measure of stone density. More specifically, HU is a linear transformation of the original linear attenuation coefficient measurement into one in which the radiodensity of distilled water at standard pressure and temperature (STP) is defined a zero HU, while the radiodensity of air at STP is −1000 HU. In addition the set may include a “Lg size” variable which is suitably set at 0 or 1 as a function of Stone Size. A “Dense Stone” variable is mapped to 0, 1, 2 . . . based on the value of HU. Another possible prediction variable is the “Stone Location” which is discretized into lower calyx, mid calyx, lower ureter, mid ureter, upper ureter, pelvis, upi and uvj in the urinary track.

The regression equation may also include one or more patient prediction variables 352. As previously mentioned, BMI (body mass index) calculated from patient height and weight, is a discriminator between Ps for the ESWL and URS treatment modalities. Variables such as Sex, Age, AntiCoag (whether patient is taking an anticoagulant) and MAC (whether patient is under Monitored Anesthesia Care and what type during the procedure) may also impact the Ps and Pc.

The regression equation may also include one or more machine prediction variables 354. For ESWL treatment, the manufacturer, make and model of the lithrotripter has been demonstrated to impact the Ps. Data is not yet available but the same may be true for URS. For example, performance may vary for Ultrasonic, Electrohydraulic or Laser treatments.

There are likely to be other prediction variables added to the regression equation as more data becomes available and is analyzed. For example, additional variables may include additional types of ESWL machines, different types of anesthesia (high frequency jet ventilation, conscious sedation), patient medical history (previous treatments, diabetic), stent inserted or removed, medical tests performed (e.g., PTT, INR, urine culture), safety pause, pre-op fluids (lasix, hydration), ureteral dilation, patient position (prone, supine, oblique), coupling solution, strapping/positioning device, respiratory excursion, timing of X-rays, ramp up protocol, and measures of shocking protocol.

Regression equations used to produce cost estimates may include cost variables such as operating room time, stent placement, ESWL revenue, etc. 356.

The regression equations for the set of probability calculations (e.g., Ps, Pc, Pi, Pn) combine a generalized linear mixed model to generate an intercept I and regression weights Wi for the prediction variables that predict a categorical outcome (e.g., success) and a conversion from the categorical outcome to a probabilistic outcome. To calculate predicted probability from a generalized linear mixed model (a model specifying random effects that predicts a categorical outcome) you must exponentiate the base of the natural logarithm, which is 2.718 (Rabe-Hesketh and Skrondal, 2008).

In an embodiment, the regression equation may be represented as follows:

Probability of Success (Ps)=X/(1+X) eqn. 1

Where X=S**(Y), Y=I+Ts is the categorical or odds model, S is a scalar equal to the natural logarithm “e” (2.718) to convert from a model that predicts a categorical outcome or odds to a model that predicts a probability, Ts is a sum of the products of stone, patient, machine prediction variable inputs and their respective regression weights Wi to success of the treatment, and I is the Intercept, which is the prediction of an outcome without the prediction variables.

The outcome model Y=I+Ts is based on certain assumptions. For example, the model may be configured so that the default conditions are most effective at producing a successful outcome. The weighted inputs reduce the effectiveness. In this case, the Intercept is biased high. Conversely, the model could be configured so that the default conditions are the least effective at producing a successful outcome and the weighted inputs improve effectiveness. In this case, the Intercept is biased low. Both are equivalent.

In an embodiment,

Ts(ESWL)=W1*size+W2*lgsize+W3*hu+W4*dense+W5*lowercalyx+W6*midcalyx+W7*lowerureter+W8*midureter+W9*upperureter+W10*pelvis+W11*upj+W12*uvj+W13*sex+W14*age+W15*bmi+W16*mac+W17*storzslxt+W276*dornierd1+W277*dornierd2+W278*dornierd3+W279*dorniersigma eqn. 2

Ts(URS)=W20*size+W21*lgsize+W22*hu+W23*dense+W24*lowercalyx+W25*midcalyx+W26*lowerureter+W27*midureter+W28*upperureter+W29*pelvis+W30*upj+W31*uvj+W32*sex+W33*age+W34*bmi+W35*mac+W36*size*hu eqn. 3

The Probability of Complications (Pc)=X/(1+X) eqn. 4

Where X=S**(I+Tc), and Tc is a sum of the products of stone, patient, machine prediction variable inputs and their respective regression weights Wi to complications of the treatment.

In an embodiment,

Tc(ESWL)=W40*size+W41*hu+W42*anticoag+W43*dense+W54*lowercalyx+W55*midcalyx+W56*lowerureter+W57*midureter+W58*upperureter+W59*pelvis+W60*upj+W61*uvj eqn. 5

Tc(URS)=W50*size+W51*hu+W52*anticoag+W53*dense+W54*lowercalyx+W55*midcalyx+W56*lowerureter+W57*midureter+W58*upperureter+W59*pelvis+W60*upj+W61*uvj eqn. 6

The Probability of infection rate (Pi)=X/(1+X) eqn. 7

Where X=S**(I+Tc), and Tc is a sum of the products of stone, patient, and treatment strategy prediction variable inputs and their respective regression weights Wi to complications of the treatment.

In an embodiment,

Tc(ESWL)=W122*size+W123*hu+W124*anticoag+W125*dense+W126*lowercalyx+W127*midcalyx+W128*lowerureter+W129*midureter+W130*upperureter+W131*pelvis+W132*upj+W133*uvj+W144*age+W145*sex+W146*mac+W147*Storzslxt+W148*dornierd1+W149*dornierd2+W150*dornierd3+W151*dorniersigma —eqn. 8

The Probability of hematoma rate (Ph)=X/(1+X) eqn. 9

Where X=S**(I+Tc), and Tc is a sum of the products of stone, patient, and treatment strategy prediction variable inputs and their respective regression weights Wi to complications of the treatment.

In an embodiment,

Tc(ESWL)=W153*size+W154*hu+W155*anticoag+W156*dense+W157*lowercalyx+W158*midcalyx+W159*lowerureter+W160*midureter+W161*upperureter+W162*pelvis+W163*upj+W164*uvj+W175*age+W176*sex+W177*mac+W178*Storzslxt+W179*dornierd1+W180*dornierd2+W181*dornierd3+W182*dorniersigma eqn. 10

The Probability of pain rate (Pp)=X/(1+X) eqn. 11

Where X=S**(I+Tc), and Tc is a sum of the products of stone, patient, and treatment strategy prediction variable inputs and their respective regression weights Wi to complications of the treatment.

In an embodiment,

Tc(ESWL)=W184*size+W185*hu+W186*anticoag+W187*dense+W188*lowercalyx+W189*midcalyx+W190*lowerureter+W191*midureter+W192*upperureter+W193*pelvis+W194*upj+W195*uvj+W206*age+W207*sex+W208*mac+W209*Storzslxt+W210*dornierd1+W211*dornierd2+W212*dornierd3+W213*dorniersigma eqn. 12

Below are calculations used to ensure that summing the probabilities of infection, hematoma, and pain equal the calculated Pc. This is relevant for users who want the categories of complications to add up to the total complication rate. This equality may not always exist from data analysis because there is the possibility that other, less frequent complications could arise and contribute to the Pc. These less frequent complications are usually too infrequent in the data registry or published literature to be used as an outcome variable in the modeling process, but they can be used when all complication types are grouped together to create Pc. The predicted probabilities for infection, hematoma, and pain can be calculated from running the equations, but displaying these probabilities so that they sum to Pc can be done by taking the each predicted rate as a proportion of Pc. To do this, once the probability algorithms for each complication type (infection, hematoma, and pain) are calculated, the following equations are run to produce the final probabilities that are displayed in the GUI.

totalcompcalculated=Pi+Ph+Pp;

infectioncompfinal=(Pi/totalcompcalculated)*Pc;

hematomacompfinal=(Ph/totalcompcalculated)*Pc; and

paincompfinal=(Pp/totalcompcalculated)*Pc. eqn. 13

A number of predicted probabilities are estimated to calculate the figures used in the cost calculations. This module displays figures in tables and graphs of elements of cost to the patient (such as likelihood of receiving a stent, requiring a secondary procedure, time in the operating room, etc.) and economic considerations to the doctor or facility (such as equipment costs, treatment time, likelihood of complications, etc.). In addition to referencing Ps and Pc, the cost calculations use probability of a stent being inserted (Pn) and predicted operating room time (Po) along with other terms to calculate the numbers displayed in the tables and graphs. Static figures taken directly from the literature and not generated or residing in predictive equations, such as average hospital stay by treatment modality, may also be included in cost calculations.

Patient economic costs may include costs in the realm of time, health risk, and financial cost. These items may comprise the probabilities and costs of a stent being inserted, operating room time, and rate of procedure retreatment. These figures can help patients may decisions on modality type preferences in areas beyond Ps and Pc. For instance, a patient may decide to select ESWL over URS because it has a lower likelihood of needing a stent and hence a second procedure to remove the stent, despite having a slightly lower Ps compared to URS.

The Probability of stent inserted (Pn)=X/(1+X) eqn. 14

Where X=S**(I+Tc), and Tc is a sum of the products of stone, patient, and treatment strategy prediction variable inputs and their respective regression weights Wi to complications of the treatment.

In an embodiment,

Tc(ESWL)=W215*size+W216*hu+W217*anticoag+W218*dense+W219*lowercalyx+W220*midcalyx+W221*lowerureter+W222*midureter+W223*upperureter+W224*pelvis+W225*upj+W226*uvj+W237*age+W238*sex+W239*mac+W240*Storzslxt+W241*dornierd1+W242*dornierd2+W243*dornierd3+W244*dorniersigma eqn. 15

The Probability of OR Time (Po)=X eqn. 16

Where X=I+Tc, and Tc is a sum of the products of stone, patient, and treatment strategy prediction variable inputs and their respective regression weights Wi to number of shocks delivered at a particular ESWL machine power level of the treatment.

In an embodiment,

Tc(ESWL)=W246*size+W247*hu+W248*anticoag+W249*dense+W250*lowercalyx+W251*midcalyx+W252*lowerureter+W253*midureter+W254*upperureter+W255*pelvis+W256*upj+W257*uvj+W268*age+W269*sex+W270*mac+W271*Storzslxt+W272*dornierd1+W273*dornierd2+W274*dornierd3+W275*dorniersigma eqn. 17

Predicted OR Time is used to populate the elements in economic cost calculations.

The cost calculations also contain economic information that is relevant to doctors and healthcare facilities. These variables include revenue, operating time, and equipment and facility costs. Providers can use this information in addition to Ps and Pc to select the optimal treatment modality for a patient and stone profile.

ESWLRevenue = 9000;
URSRevenue = 6700;
URSpredictedORTime = Po + Po * .667;
SWLpredictedORCost = Po * 20;
URSpredictedORCost = (Po + Po * .667) * 20;
ESWLequipmentcost = 1200;
URSequipmentcost = 900 + 1400;
ESWLPredictedRetreatmentRate = 1 − Ps
URSPredictedRetreatmentRate = 1 − Ps
ESWLPredictedComplicationRate = Pc
URSPredictedComplicationRate = Pc

SWL

| | |
|---|---|
| Revenue | |
| Treatment | A |
| Retreatment | B |
| Total average | A + B |
| Costs | |
| Equipment | C |
| OR | D |
| Complication costs | E |
| Retreatment costs | F |
| Total average | C + D + E + F |
| Gross Profit | A + B − (C + D + E + F) |

URS

| | |
|---|---|
| Revenue | |
| Treatment | A1 |
| Retreatment | B1 |
| Total average | A1 + B1 |
| Costs | |
| Equipment | C1 |
| OR | D1 |
| Complication costs | E1 |
| Retreatment costs | F1 |
| Total average | C1 + D1 + E1 + F1 |
| Gross Profit | A1 + B1 − (C1 + D1 + E1 + F1) |

A ESWLRevenue
B ESWLRevenue * ESWLpredictedretreatmentrate
C ESWLequipmentcost
D ESWLpredictedORcost
E (ESWLpredictedcomplicationrate * (ESWLequipmentcost + ESWLpredictedORcost)
F (ESWLPredictedRetreatmentRate * (ESWLequipmentcost + ESWLpredictedORcost)
A1 URSRevenue
B1 URSRevenue * URSpredictedretreatmentrate
C1 URSequipmentcost
D1 URSpredictedORcost
E1 (URSpredictedcomplicationrate * (URSequipmentcost + URSpredictedORcost)
F1 (URSPredictedRetreatmentRate * (URSequipmentcost + URSpredictedORcost)

The regression weights for the prediction variables are coded for the regression equation. In general, the weights may be fixed scalar values, functions of other prediction variables, conditional probabilities based on a Bayesian model or other. The weights may provide for linear or non-linear combinations of the prediction variables.

In an embodiment, the prediction variables are coded according to:

Size: Use inputted value of stone size

Lgsize: If size>=TH0 set lgsize=1, else lgsize=0 Hounsfield Units (HU): Use inputted value.

Dense: If HU<TH1 then code dense to 0; If value is >=T1 and <TH2 then code dense to 1; If input is >=TH2 then code dense to 2.

Stone Location: Use inputted binary value of 0 or 1 (only one has a value 1)

Sex: Male=1; Female=0

Age: Use input Value

BMI: Use value computed from height and weight

MAC: if Monitored Anesthesia Care=1 then mac=1. Since MAC and general anesthesia are the only anesthesia methods modeled, if mac=0 then general anesthesia is assumed. This is a result of dummy coding the anesthesia variable and aligns with the other binary input variables in the model such as sex and anticoag.

ESWL Machine: Use inputted binary value, and

Anticoag: yes=1, no=0.

Additional variables used to produce cost calculations are as follows:

Operating room time: predicted from above variables

Stent placement: predicted from above variables or used from academic literature

ESWL revenue: estimated from industry data

URS revenue: estimated from industry data

ESWL equipment cost: estimated from industry data

URS equipment cost: estimated from industry data

ESWL retreatment rate: predicted from above variables

URS retreatment rate: predicted from above variables

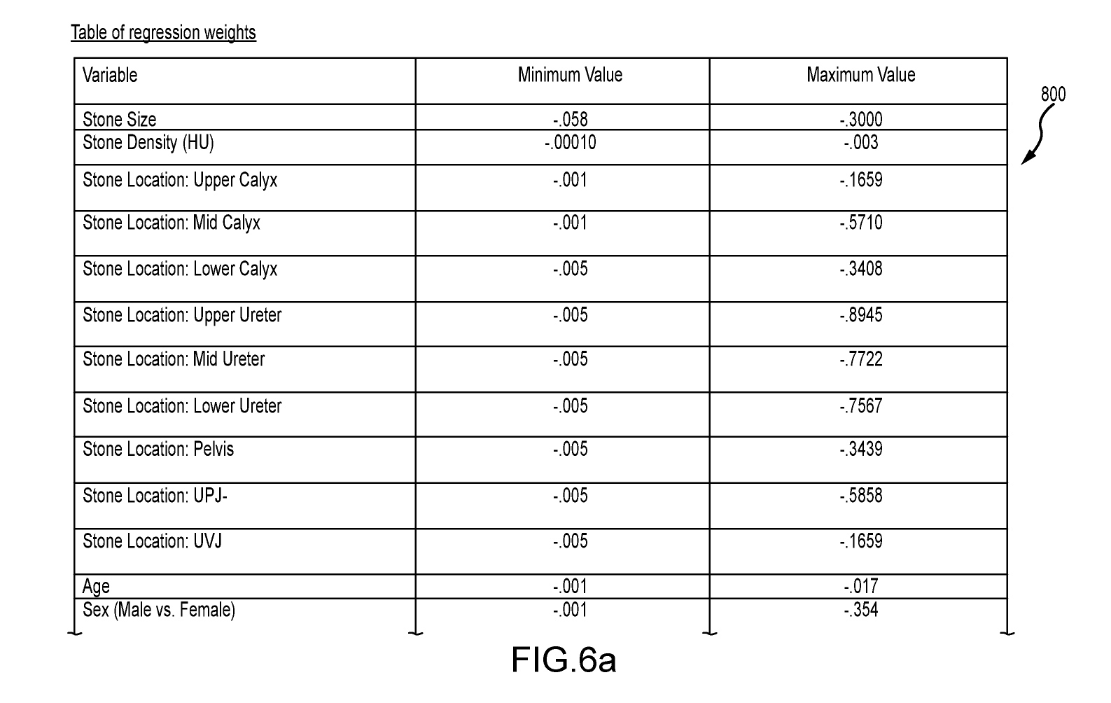

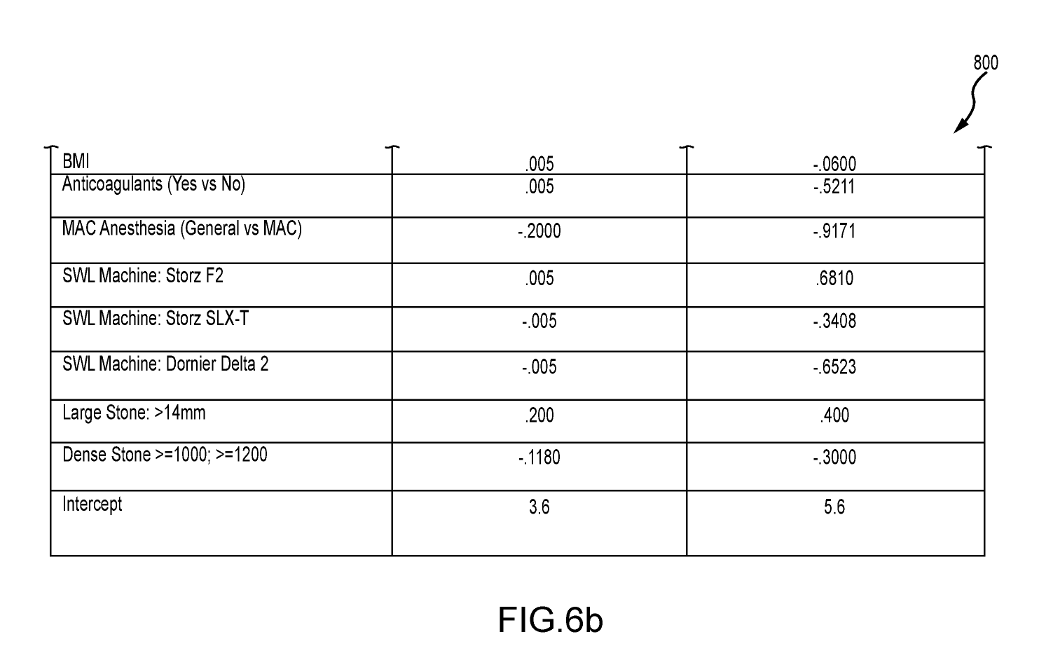

Referring now to FIGS. 6a and 6b, an embodiment of a Table 800 of regression weights includes a minimum and a maximum regression weight for each of the prediction variables. These specific weights are based upon the assumption that the outcome model is configured so that the optimal level or value of each input variable is contained in the intercept and that each time a non-optimal level or value is selected (e.g., MAC anesthesia over general anesthesia) the regression weight of this value will subtract from the intercept and lead to a reduction in the calculation probability of success. The range of regression weights covers the weights for both the ESWL and URS procedures and the 50^{th} percentile and the literature benchmark for each in relation to both treatment success (Ts) and treatment complications (Tc).

The specific value of a given weight is dependent on many factors including: treatment modality, combination of prediction variables in the regression equation, and the starting point of the intercept. Furthermore, each regression weight should be considered in absolute value because the weighted prediction of the predictor variables can either be designed to subtract from a high intercept (i.e., start with the assumption of default optimal input values and subtract from this intercept when non-optimal values are selected) or add from a low intercept (i.e., start with the assumption of default of least optimal input values and add to this intercept when optimal values are selected). Subtracting regression weights from a high intercept or adding regression weights to a low intercept will not change the final calculated probability.

One of ordinary skill in the art will understand that the specific values of the weights and even the range of the weights may change as different combinations of prediction variables are implemented, as additional information is gathered on individual prediction variables and as new techniques are incorporated.

In one approach, the regression weights for a given treatment modality are very similar for the benchmark and 50th percentile cases. The data analytics provided by the 50^{th} percentile essentially sets the regression weights. The difference in outcome effectiveness or probability between benchmark and the 50^{th} percentile is accounted for in a difference in the Intercept values. If specific values for regression weights were available in the literature, different weights could be used.

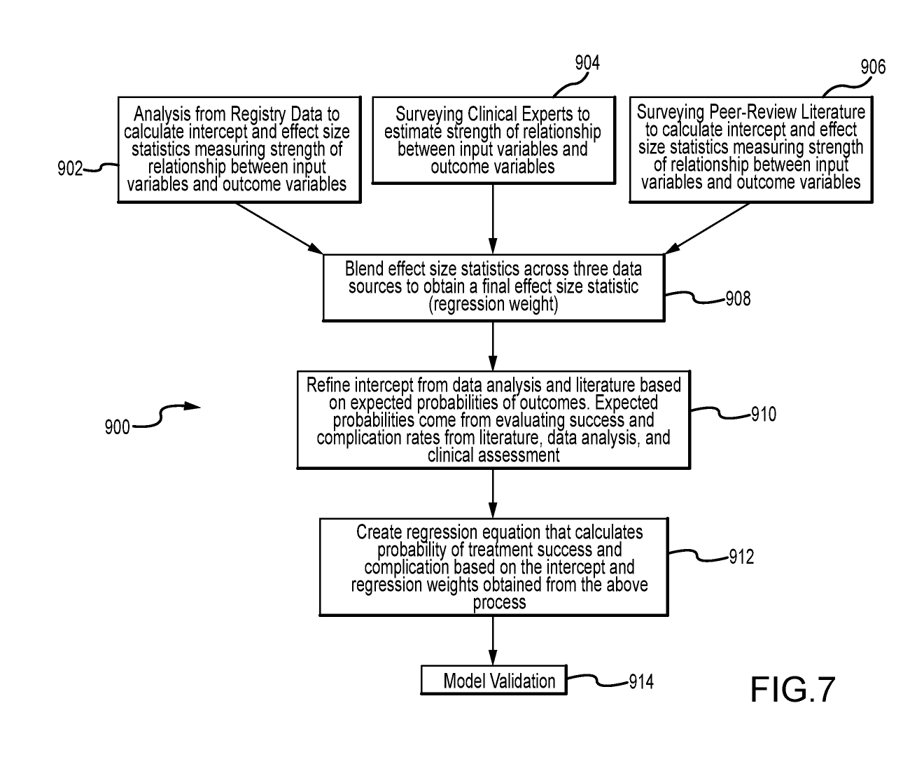

FIG. 7 illustrates an embodiment of a method 900 for defining the regression equation (e.g., the prediction variables) and determining the regression weights and intercept for the equation.

Step 1: Identifying Relevant Variables and Obtaining Regression Weights for each:

1. Clinical Assessment 904: a team of urologists and ESWL technologists were tasked to indicate all relevant variables that can affect treatment success and treatment complications. Once this list of relevant variables was created, the variables were grouped into categories such as doctor dependent, technologist dependent, stone characteristics, patient demographics, etc. A path model was specified to indicate causal relationships among these relevant variables on the outcomes of treatment success (stone fragmentation) and treatment complications (bleeding, ER visit, etc.). Once this path model was specified, the team of urologists and technologists estimated the strength of these variables on these outcomes in categories of weak, moderate, and strong. A final strength category of weak, moderate, or strong was given to each relationship or path once all team members agreed on this strength characterization.
2. Data Analytics 902: Statistical models were run to identify which variables in our database were related to treatment success and complications and estimate their regression weights (i.e., strength of association) on these outcomes. This process entailed including patient and stone characteristics that were collected on the International Stone Registry encounter form as predictors in a generalized linear mixed model with treating physician and patient specified as random effects. This statistical model produces and comprises the regression weights. Treatment success (fragments less than or equal to 4 mm) and treatment complications (post-op bleeding, ER visit, etc.) were measured from follow-up surveys administered to the physician. Inclusion criteria for the analysis were as follows: follow-up survey must have been completed with a known outcome (image taken at follow-up), patient must have been seen for a follow-up appointment within 60 days of the initial treatment, and single stone treatments.
3. Literature review 906: Peer-reviewed academic literature was reviewed to identify the variables that are related to treatment success and complications and the reported regression weights for each. Literature was obtained by searching Pubmed with keywords relating to SWL and URS success and complications. Keywords were included relating to specific variables known to affect outcomes such as stone size, Hounsfield Units, BMI, etc.). Literature used to create the American Urological Association Surgical Management of Stone Guidelines were also included in this review. Articles were reviewed and the reported regression weights or odds ratios were collected in an Excel sheet. Patterns in the reported regression weights were identified and based on convergence of numbers among the published articles. A single, final regression weight was selected to use to represent the literature regression weight. For variables with significant variation in regression weights across studies, an average weight was constructed by weighting regression weights more heavily from studies that had larger sample sizes and those that included other relevant variables such as stone size, density, and location.

Step 2: Blending final regression weights 908 (strengths of association between predictor and outcome):

The strengths of association obtained from data analysis, literature review, and clinical assessment are blended to come up with a final regression weight for each predictor-outcome relationship. Merging these three sources is intended to generate the most accurate regression weights for the largest number of circumstances. This generalist approach is important so that anyone in the world can enter patient and stone characteristics in the interface and obtain probabilities of success that are most likely to be correct for them, as opposed to only modeling regression weights for a specific population. The blending process is initiated by taking regression weights for each variable from the International Stone Registry data analysis and the literature. Since very few regression weights for the particular input variables are not present in the literature, most regression weights were generated from data analysis and then the intercept was adjusted to align with success and complication rates reported in the literature. If a single regression weight was reported in the literature or regression weights were reported in the literature for different categorizations (such as stone size < or >10 mm instead of reporting weights for stone size on a continuous scale, then these categorized regression weights were used to validate those obtained from data analysis. In these cases, literature benchmarks meant that literature was reviewed and 1) whatever regression weights were reported were gauged in order to validate the use of the data-derived weights and 2) their reported outcomes were used to estimate what the calculated probabilities should end up as.

Once a single, final regression weight was obtained from the literature and from data analysis, these two weights were blended to derive a single, final regression weight for each input variable. Once these final, blended weights were selected, they were entered into a predictive model that transformed them to calculate probabilities of success and complications based on input values.

Step 3: Estimate Intercept 910

1. Estimate Intercept from data analysis and then refine based on expected probabilities of outcomes. Expected probabilities come from evaluating success and complication rates from literature, data analysis, and clinical assessment. Based on convergence or similarity of regression weights between the literature and those obtained from the International Stone Registry, differences in overall success rates between these two sources were attributed to be due to different intercepts and not regression weights of particular variables. In other words, in comparing the International Stone Registry regression weights with those reported in the literature it was assumed that the relationship between stone size and success rate, for example, does not really differ between these two data sources. However, what was different was the absolute reported success rate, which would be shown as a difference in intercept. Once the regression weights that most closely characterized those obtained from the literature and data analysis were characterized, these numbers, along with the intercepts were adjusted based on the absolute success rates reported in the literature, data analysis, and clinical assessment. This process ensured that each regression weight and the intercept would produce the expected outcome for a particular patient and stone demographic. Defining the expected outcome was accomplished in the following manner: for International Stone Registry data analysis, the success rate based on the International Stone Registry data for certain profiles was run such a lower ureter stone that was 8 mm on a 55 year old patient with a BMI of 23. The intercepts and regression weights in the algorithm were adjusted to produce predicted success rates that align with the observed success rate from the International Stone Registry data; for clinical assessment, the intercepts and regression weights in the algorithm were adjusted to produce what urologists expected to see for various patient and stone demographics. Finally, the literature was used to ensure that changes in levels of predictor variables (<10 mm stone vs >10 mm stone) align with observed changes in success rate. This method was used to ensure that the blended intercepts and regression weights and the corresponding algorithm produced success rates comparable to those observed in the International Stone Registry data, the literature, and clinical assessment.

Step 4: Create the regression equation 912

1. Create regression algorithm that calculates probability of treatment success and complications based on the intercept and regression weights obtained from the above process.

Step 5: Model Validation 914

1. Standard research and statistical methods were used to validate the algorithm. The algorithm was run on a random sample of SWL treatment performed a random 70/30 split of 2,100 SWL treatment records for renal and ureteral stones from 2010-2016 to validate a generalized linear mixed model (GLMM) using statistical software (PROC GLIMMIX in SAS 9.4). This model was a good predictor of success in the validation dataset, AUC=0.81. The training model was also significantly related to complications, LR χ²=538.75, p<0.0001, AUC=0.91. This model was a fair predictor of complication rate in the validation dataset, AUC=0.77.

As an example, a generalized linear mixed model was run that specified all the inputs as predictor variables and urologist as a random effect. The outcome variable was success rate (stone free or fragments <=4 mm). The observed stone size regression weight was −0.2340.

All of the regression weights for stone size that were published in the peer-reviewed literature were compiled. From this list, a weighted average of regression weights was estimated giving greater weight to studies with larger sample sizes and studies that also included variables of Hounsfield Units, BMI or skin to stone distance, and stone location. This final, estimated weighted average was compared with the −0.2340 produced from the data analysis. An estimated weighted average of the data analysis regression weight and the literature regression weight was calculated.

A team of urologists and technologists was surveyed to estimate the strength of the relationship between input variable and outcome on a scale of strong, moderate, and weak. For stone size, this team unanimously agreed that the relationship between stone size and treatment success was strong. This finding was in accord with the large regression weight obtained through data analysis and literature review. As such, there was no need to adjust the regression weights from data analytics and literature review.

Once this final regression weight was selected, an intercept was established that would produce the expected probability of success for stone sizes based on what was seen in the literature, International Stone Registry, and clinical assessment. Expected success probabilities were created by looking at success rates reported in the literature for stone sizes (usually reported as <=10 mm and >10 mm in the literature), by running success rates for each stone size in the International Stone Registry, and by asking experienced urologists what they would expect a probability of success to be for each stone size. Using these three numbers as a guide, the intercepts were iteratively adjusted so that particular stone sizes would produce probabilities of success that align with these expectations of the literature and observed probabilities in the International Stone Registry.

While several illustrative embodiments of the invention have been shown and described, numerous variations and alternate embodiments will occur to those skilled in the art. Such variations and alternate embodiments are contemplated, and can be made without departing from the spirit and scope of the invention as defined in the appended claims.

We claim:

1. A graphical user interface (GUI) driven computer implemented system for comparison and selection of treatment modalities for surgical management of stone disease configured for display on a display screen of an electronic device, comprising:
computer memory configured to store regression weights for a plurality of stone prediction variables for each of an extracorporeal shock wave lithotripsy (ESWL) and a Ureteroscopy (URS) treatment modality;
a computer processer configured to compute a probability of success (Ps) and a probability of complications (Pc) for the ESWL and URS treatment modalities as a weighted combination of said regression weights and input values for said prediction variables; and
instructions in said computer memory executable by said computer processor to display and operate a GUI, said GUI comprising a first user selectable display space and a first results display space configured for simultaneous display on the display screen of the electronic device, wherein the first user selectable display space is configured to display the plurality of stone prediction variables and receive and display input values for each said stone and patient prediction variable and the first results display space is configured to display the computed Ps and Pc for each of ESWL and URS.

2. The GUI driven computer implemented system of claim 1, wherein said stone prediction variables include at least stone size for both ESWL and URS treatment modalities.

3. The GUI driven computer implemented system of claim 2, wherein the Ps for the URS treatment modality includes a weighted product of the stone size and a stone density or a nonlinear term for either stone size or stone density.

4. The GUI driven computer implemented system of claim 2, wherein the absolute value of regression weights for stone size lie between 0.058 and 0.3.

5. The GUI driven computer implemented system of claim 2, wherein said stone prediction variables further include stone locations for both ESWL and URS treatment modalities.

6. The GUI driven computer implemented system of claim 2, wherein said computer memory is configured to store regression weights for a plurality of patient prediction variables including at least BMI and anesthesia for the ESWL treatment modality.

7. The GUI driven computer implemented system of claim 6, wherein the plurality of patient prediction variables further includes one or more medical history prediction variables.

8. The GUI driven computer implemented system of claim 2, wherein computer memory configured to store regression weights for at least one ESWL machine for the ESWL treatment modality.

9. The GUI driven computer implemented system of claim 2, wherein said stone prediction variables further include stone location, wherein Ps is a weighted combination of stone location for both ESWL and URS and Pc is a weighted combination of stone location for URS.

10. The GUI driven computer implemented system of claim 9, wherein Pc is further a weighted combination of stone size and a stone density or non-linear terms for either stone size or stone density for both ESWL and URS.

11. The GUI driven computer implemented system of claim 1, wherein said stone prediction variables include stone size, large size, Hounsfield Units (HU), stone density and stone location, a plurality of patient prediction variables include sex, age, BMI, anti coagulants and anesthesia and a machine prediction variable includes ESWL machine type.

12. The GUI driven computer implemented system of claim 11, wherein the Ps is computed as X/(1+X) where X=S**(I+Ts) and Pc is computed as Y/(1+Y) and Y=S**(I+Tc) where is S is approximately 2.7, I is an intercept and
Ts(ESWL)=W1*size+W2*lgsize+W3*hu+W4*dense+W5*lowercalyx+W6*midcalyx+W7*lowerureter+W8*midureter+W9*upperureter+W10*pelvis+W11*upj+W12*uvj+W13*sex+W14*age+W15*bmi+W16*mac+W17*storzslxt+W148*dornierd1+W149*dornierd2+W150*dornierd3+W151*domiersigma
Ts(URS)=W20*size+W21*lgsize+W22*hu+W23*dense+W24*lowercalyx+W25*midcalyx+W26*lowerureter+W27*midureter+W28*upperureter+W29*pelvis+W30*upj+W31*uvj+W32*sex+W33*age+W34*bmi+W35*mac+W36*size*hu,
Tc(ESWL)=W40*size+W41*hu+W42*anticoag+W43*dense+W54*lowercalyx+W55*midcalyx+W56*lowerureter+W57*midureter+W58*upperureter+W59*pelvis+W60*upj+W61*uvj, and
Tc(URS)=W50*size+W51*hu+W52*anticoag+W53*dense+W54*lowercalyx+W55*midcalyx+W56*lowerureter+W57*midureter+W58*upperureter+W59*pelvis+W60*upj+W61*uvj,
wherein absolute values are:
W1, W20, W40 and W50 lie between 0.058 and 0.3,
W2 and W21 lie between 0.2 and 0.4,
W3, W22, W41 and W51 lie between 0.0001 and 0.003,
W4, W23, W43 and W53 lie between 0.1180 and 0.30,
W5, W24 and W54 lie between 0.005 and 0.3408,
W6, W25 and W55 lie between 0.001 and 0.571,
W7, W26, and W56 lie between 0.005 and 0.7567,
W8, W27 and W57 lie between 0.005 and 0.7722,
W9, W28 and W58 lie between 0.005 and 0.8945,
W10, W29 and W59 lie between 0.005 and 0.3439,
W11, W30 and W60 lie between 0.005 and 0.5858,
W12, W31 and W61 lie between 0.005 and 0.1659,
W13 and W32 lie between 0.001 and 0.354,
W14 and W33 lie between 0.001 and 0.017,
W15 and W34 lie between 0.005 and 0.06,
W16 and W35 lie between 0.2 and 0.9171,
W17 lies between 0.005 and 0.3408, and
W148, 149, 150, 151, 179, 180, 181, 182, 210, 211, 212, 213, 241, 242, 243, 244, 272, 273, 274, 275, 276, 277, 278, 279 lies between 0.005 and 0.6523

13. The GUI driven computer implemented system of claim 1, wherein the Ps and Pc are computed and displayed for at least a percentile grouping defined in terms of performance success rate.

14. The GUI driven computer implemented system of claim 13, wherein the Ps and Pc are computed and displayed for a first percentile grouping corresponding to an average performance success rate and a second grouping corresponding to an above average performance success rate.

15. The GUI driven computer implemented system of claim 13, wherein the Ps and Pc are computed and displayed for a benchmark grouping

16. The GUI driven computer implemented system of claim 15, wherein the regression weights are determined from data analytics and verified against literature and clinical assessment, wherein an intercept value or differences in regression weights accounts for differences in successful outcomes between the benchmark and percentile grouping.

17. The GUI driven computer implemented system of claim 1, further comprising:
a second user selectable display space configured to display a calculate button user selection of which initiates the computer processor to compute the Ps and Pc.

18. The GUI driven computer implemented system of claim 1, further comprising instructions in said computer memory to compute and display a patient economic cost based on the input values for the patient and stone prediction variables.

19. The GUI driven computer implemented system of claim 1, further comprising instructions in said computer memory to compute and display a provider economic cost based on the input values for the patient and stone prediction variables.

20. A graphical user interface (GUI) driven computer implemented system for comparison and selection of treatment modalities for surgical management of stone disease configured for display on a display screen of an electronic device, comprising:
computer memory configured to store regression weights for a plurality of stone prediction variables including at least stone size, stone density and stone location and at least one patient prediction variable for each of a ESWL and a Ureteroscopy (URS) treatment modality for a percentile grouping defined in terms of performance success rate;
a computer processer configured to compute a probability of success (Ps) and a probability of complications (Pc) and a patient or provider economic cost for the ESWL and URS treatment modalities for the percentile grouping as a weighted combination of said regression weights and input values for said prediction variables; and
instructions in said computer memory executable by said computer processor to display and operate a GUI, said GUI comprising a first user selectable display space and at least one results display space configured for simultaneous display on the display screen of the electronic device, wherein the first user selectable display space is configured to display the stone, patient, and ESWL machine prediction variables and receive and display input of values for each said stone and patient prediction variable, and a first results display space configured to display the computed Ps and Pc for the percentile grouping and a second results display space configured to display the patient or provider economic cost for the percentile grouping.
